(19) United States
(12) Patent Application Publication
(10) Pub. No.: US 2014/0017185 A1
(43) Pub. Date: Jan. 16, 2014

(54) Hair Care Agent

(71) Applicants: Matthias Hloucha, Koln (DE); Jasmin Schorb, Monheim (DE); Hans-Martin Haake, Erkrath (DE); Werner Seipel, Hilden (DE); Marc Beuche, Vauhallan (FR); Cristina Amela Conesa, Cerdanyola del Valles (ES); Stephane Lacoutiere, Gif Sur Yvette (FR); Guadalupe Pellon, Duesseldorf (DE)

(72) Inventors: Matthias Hloucha, Koln (DE); Jasmin Schorb, Monheim (DE); Hans-Martin Haake, Erkrath (DE); Werner Seipel, Hilden (DE); Marc Beuche, Vauhallan (FR); Cristina Amela Conesa, Cerdanyola del Valles (ES); Stephane Lacoutiere, Gif Sur Yvette (FR); Guadalupe Pellon, Duesseldorf (DE)

(73) Assignee: Cognis IP Management GmbH, Dusseldorf (DE)

(21) Appl. No.: 14/009,046

(22) PCT Filed: Mar. 23, 2012
(86) PCT No.: PCT/EP2012/001270

§ 371 Date: Sep. 30, 2013

(30) Foreign Application Priority Data
Apr. 1, 2011 (EP) 11160780

(51) Int. Cl. A61K 8/06; A61Q 5/12

(52) U.S. Cl. CPC A61K 8/068; A61Q 5/12
USPC 424/59; 424/70.27; 424/70.28; 424/70.19; 424/70.9

(57) ABSTRACT

The invention relates to a conditioner in the form of an optically non-transparent dispersion containing at least one cationic surfactant, a micro emulsion containing bl) at least one alkyl glycoside and/or an alkyl oligoglycoside, b2) at least one cosurfactant which does not fall under the definition of bl), b3) an organic oil phase, and b4) water, at least one fatty alcohol, optionally further surfactants and optionally further cosmetic additives, wherein the sum of all the surfactants present in the conditioner makes up a proportion of at most 10 wt % of the conditioner. The invention also relates to the use and production thereof.

The present invention relates to a conditioner in the form of an optically non-transparent dispersion comprising at least one cationic surfactant, a microemulsion comprising b1) at least one alkyl glycoside and/or alkyl oligoglycoside, b2) at least one cosurfactant which does not fall under the definition of b1), b3) an organic oil phase, and b4) water, at least one fatty alcohol, optionally further surfactants, and optionally further cosmetic additives, where the sum of all of the surfactants present in the conditioner constitute a fraction of at most 10% by weight of the conditioner, as well as to its use and preparation.

So-called conditioners are hair care compositions which are used for conditioning hair. They are for example brought into contact with the hair after hair washing in the form of a rinse.

In contrast to shampoos, conditioners comprise a relatively small fraction of surfactants since they are not used for the washing, but for the care, in particular conditioning, of the hair. Typically, conditioners are in the form of optically non-transparent dispersions in which, for example, fatty alcohols or waxes are present dispersed in an aqueous matrix.

After washing, hair often feels rough and brittle, particularly if it has already been damaged by environmental influences. Moreover, hair can also be damaged by coloring or perming and it is then often characterized after hair washing by a dry straw-like feel.

For this reason, conditioning agents called are often used in hair care compositions which are supposed to counteract these disadvantages. The conditioning agents can be used in shampoos, they can also be used in the aforementioned conditioners. Hair care compositions are often found which comprise silicones as conditioning agents. However, these can sometimes attach irreversibly to the hair and thus cause, for their part, negative effects on the feel, and in the worst case scenario they even lead to problems during the coloring and perming of hair. It is therefore desirable to provide hair care agents, in particular conditioners, which also make do without silicones and are nevertheless effective.

Furthermore, oils and waxes are suitable as conditioning agents in these cosmetic preparations. However, these are in no way as pronounced in their effect as the aforementioned silicones. Moreover, by using these conditioning agents, only cloudy formulations are frequently possible and/or these oils and waxes can often only be stabilized in the preparations in small amounts.

Small particle sizes of the dispersed phase contribute in emulsions to particularly strong interactions with surfaces such as e.g. hair, since the surface-to-volume ratio is particularly large. Furthermore, they contribute to good sensory properties and good compatibility.

Shampoo compositions which comprise silicones are known in the prior art. For example, EP 1722860 describes a shampoo composition comprising anionic surfactants, a microemulsion of silicones and cationic polymers.

Cosmetic preparations are also known which, besides alkoxylated surfactants, include microemulsions which comprise silicone oils and are based on anionic surfactants. EP 0529883 describes preparations which comprise a cationic deposition polymer alongside lauryl ether sulfate and betaine. A disadvantage here is that only silicone oils can be used and alkoxylated surfactants are present.

By using alkoxylated surfactants, mostly alkyl ether sulfates, skin irritations can arise when using the cosmetic compositions and, moreover, the calls for “green cosmetics” which are free from alkoxylated compounds are increasing. For these limitations with regard to the surfactants, there is hitherto no satisfactory solution for providing hair care compositions with good conditioning performance.

WO 2008/155075 describes cosmetic preparations which, besides non-alkoxylated surfactants, comprise a microemulsion and at least one cationic polymer. These preparations are used as conditioning agents in shampoo and hair treatment compositions. For a better conditioning effect, a cationic polymer has to be mandatorily used. The content of surfactants is up to 20% by weight. These large amounts of surfactants can lead to skin irritations, particularly in the case of leave-on products such as e.g. hair conditioners.

Cosmetics & Toiletries magazine, volume 124, number 5, May 2008, pages 58 to 69 discloses that shampoos which comprise microemulsions have a good conditioning effect on hair. Conditioners comprising microemulsions are not disclosed.

The object of the present invention is to provide a conditioner which has a good conditioning performance, in particular improves the combability of the hair. In this connection, this good conditioning performance should be achieved without necessarily having to use silicones (which belong to the silicon compounds) or alkoxylated ingredients.

This object is achieved by a conditioner in the form of an optically non-transparent dispersion comprising
A) at least one cationic surfactant,
B) a microemulsion comprising
   b1) at least one alkyl glycoside and/or alkyl oligoglycoside,
   b2) at least one cosurfactant which does not fall under the definition of b1),
   b3) an organic oil phase, and
   b4) water,
C) at least one fatty alcohol,
D) optionally further surfactants which do not fall under the definition of A) or C),
E) optionally further cosmetic additives which do not fall under the definition of A), C) or D),
   where the sum of all of the surfactants present in the conditioner constitute a fraction of at most 10% by weight of the conditioner.

Optically non-transparent dispersion means that the conditioner appears cloudy or milky when viewed with the naked eye and not for example transparent like a microemulsion. In this connection, dispersion is understood as meaning either emulsion (a liquid phase dispersed in a second liquid phase) or else suspension (a solid phase dispersed in a liquid phase).

The conditioner according to the invention is thus in the form of a dispersion, where firstly finely divided, water-immiscible particles which comprise the components b1), b2) and b3) are dispersed in a continuous aqueous phase. These particles are so small that the conditioner would be present as a transparent microemulsion if no other dispersed particles were present. However, in the conditioner according to the invention, larger particles are furthermore dispersed which comprise inter alia the at least one fatty alcohol C). As a result, the conditioner appears optically non-transparent overall. As a consequence of production, a certain exchange of the components of the conditioner can naturally take place, e.g. a certain amount of oil phase b3) can turn into the large dispersed particles, or a certain amount of fatty alcohol C) can turn into the small dispersed particles.

The conditioner according to the invention comprises a continuous aqueous phase. The water present therein can, particularly if the preparation takes place such that a microemulsion is combined with the other components which can likewise include water, originate from the microemulsion or from the other components. Once combined, only a water phase is then present and it cannot be differentiated from where the water originated. In this respect, the water referred to in claim 1 with b4) thus means water which can originate from the microemulsion optionally used for producing the conditioner or which can originate from the other components. The same is true for the quantitative data in claim 5, in which the water fractions from b4) and F) add up to a total amount of water, irrespective of where they originate from.

Particular embodiments of the conditioner according to the invention are given by the subjects of the claims dependent on claim 1.

Further subjects of the present invention are the use of the conditioner according to the invention and the preparation of the conditioner according to the invention according to the corresponding patent claims.

As component A, the conditioners according to the invention comprise cationic surfactants. Cationic surfactants which can be used are in particular quaternary ammonium compounds of the formula I and/or II.

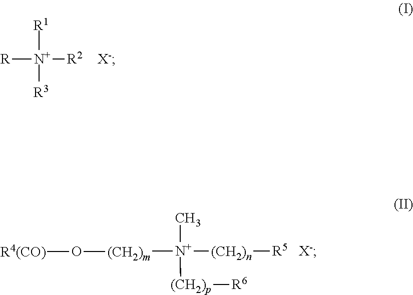

where in (I) R and R¹, independently of one another, are linear or branched alkyl and/or alkenyl radicals having 6 to 22, preferably 12 to 18, carbon atoms, R² is a saturated C1-C4 alkyl or hydroxyalkyl radical, R³ is either R, R¹ or R² or is an aromatic radical. X is either a halide, methosulfate, methophosphate or phosphate ion, and mixtures of these. Examples of cationic compounds of the formula (I) are didecyl-dimethylammonium chloride, ditallowedimethylammonium chloride or dihexadecylammonium chloride. Typical examples are also hexadecyltrimethylammonium chloride or hydroxyethyl hydroxycetyl dimonium chloride.

Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Furthermore, the very readily biodegradable quaternary ester compounds, such as, for example, dialkylammonium methosulfates and methylhydroxyalkyldialkoyloxyalkyl-ammonium methosulfates and the corresponding products of the Dehyquart® series such as dicocoylethylhydroxy-ethylmonium methosulfate, dipalmitoylethylhydroxyethyl-monium methosulfate, can be used as cationic surfactants.

Compounds of the formula (II) are so-called ester quats. Ester quats are characterized by excellent biodegradability. They are able to impart a particular soft feel to the conditioner according to the invention. They are known substances which are prepared by the relevant methods of organic chemistry. Here, R⁴ is an aliphatic alkyl radical having 12 to 22 carbon atoms with 0, 1, 2 or 3 double bonds; R⁵ is H, OH or O(CO)R⁷, R⁶ is, independently of R⁵, H, OH or O(CO)R⁸, where R⁷ and R⁸, independently of one another, are in each case an aliphatic alkyl radical having 12 to 22 carbon atoms with 0, 1, 2 or 3 double bonds. m, n and p can in each case, independently of one another, have the value 1, 2 or 3. X can either be a halide, methosulfate, methophosphate or phosphate ion, and mixtures of these. Preference is given to compounds which comprise for R⁵ the group O(CO)R⁷ and for R⁴ and R⁷ alkyl radicals having 16 to 18 carbon atoms. Particular preference is given to compounds in which R⁶ is moreover OH. Examples of compounds of the formula (II) are methyl-N-(2-hydroxyethyl)-N,N-di(tallowacyloxyethyl)-ammonium methosulfate, bis(palmitoyl)ethylhydroxyethyl-methylammonium methosulfate, N-methyl-N(2-hydroxy-ethyl)-N,N-(dioleoylethyl)ammonium methosulfate or methyl-N,N-bis(acyloxyethyl)-N-(2-hydroxyethyl)ammonium methosulfate. If quaternized compounds of the formula (II) which have unsaturated alkyl chains are used, preference is given to the acyl groups whose corresponding fatty acids have an iodine number between 5 and 80, preferably between 10 and 60 and in particular between 15 and 45 and which have a cis/trans isomer ratio (in % by weight) of greater than 30:70, preferably greater than 50:50 and in particular greater than 70:30. Standard commercial examples are methyl-hydroxyalkyldialkoyloxyalkylammonium methosulfates or the products from Cognis known under Dehyquart™.

Typical further examples of ester quats which can be used within the context of the invention are products based on caproic acid, caprylic acid, capric acid, lauric acid, myristic acid, palmitic acid, isostearic acid, stearic acid, oleic acid, elaidic acid, arachic acid, behenic acid and erucic acid, and also technical-grade mixtures thereof, as are produced for example during the pressurized cleavage of natural fats and oils. Preference is given to using technical-grade C12/18-coconut fatty acids and in particular partially hydrogenated C16/18-tallow or palm fatty acids, and also elaidic acid-rich C16/18-fatty acid cuts. For producing the quaternized esters, the fatty acids and the triethanolamine can be used in the molar ratio of 1.1:1 to 3:1. With regard to the application properties of the ester quats, a use ratio of 1.2:1 to 2.2:1, preferably 1.5:1 to 1.9:1 has proven to be particularly advantageous. The preferred ester quats are technical-grade mixtures of mono-, di- and triesters with an average degree of esterification of 1.5 to 1.9 and are derived from technical-grade C16/18-tallow or palm fatty acid.

Besides the quaternized fatty acid triethanolamine ester salts, suitable ester quats are in addition also quaternized ester salts of fatty acids with diethanolalkylamines. Finally, a further group of suitable ester quats to be mentioned are the quaternized ester salts of fatty acids with 1,2-dihydroxypropyldialkylamines. Furthermore, suitable ester quats are also substances in which the ester bond is replaced by an amide bond, preferably based on diethylenetriamine.

Besides the compounds of the formulae (I) and (II), short-chain, water-soluble, quaternary ammonium compounds can also be used, such as trihydroxyethyl-methylammonium methosulfate or the alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride.

Further preferred compounds are the diester quats of the formula (III) which, besides the softness, also provide for stability and color protection in the case of colored hair. R²¹ and R²² here are, independently of one another, in each case an aliphatic radical having 12 to 22 carbon atoms with 0, 1, 2 or 3 double bonds.

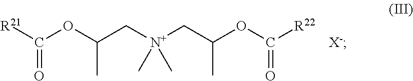

Besides the quaternary compounds described above, it is also possible to use other known compounds, such as, for example, quaternary imidazolinium compounds of the formula (IV), where R⁹ is H or a saturated alkyl radical having 1 to 4 carbon atoms, R²² and R¹¹, independently of one another, are in each case an aliphatic, saturated or unsaturated alkyl radical having 12 to 18 carbon atoms, R¹⁰ can alternatively also be O(CO)R²⁰, where R²⁰ is an aliphatic, saturated or unsaturated alkyl radical having 12 to 18 carbon atoms, and Z is an NH group or oxygen, and X is an anion. q can assume whole-numbered values between 1 and 4.

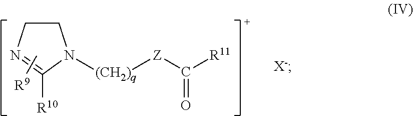

Further suitable quaternary compounds are described by formula (V), where R¹², R¹³ and R¹⁴, independently of one another, are a C1-4-alkyl, alkenyl or hydroxyalkyl group, R¹⁵ and R¹⁶, in each case selected independently, are a C8-28-alkyl group, and r is a number between 0 and 5.

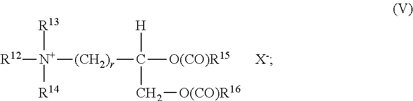

Protonated alkylamine compounds, and also the non-quaternized, protonated precursors of the cationic emulsifiers are also suitable.

Further cationic surfactants which can be used according to the invention are the quaternized protein hydrolyzates.

To produce the ester quats, it is possible to start from either fatty acids or else the corresponding triglycerides. It is likewise possible to carry out the condensation of the alkanolamines with the fatty acids in the presence of defined amounts of dicarboxylic acids, such as e.g. oxalic acid, malonic acid, succinic acid, maleic acid, fumaric acid, glutaric acid, adipic acid, sorbic acid, pimelic acid, azelaic acid, sebacic acid and/or dodecanedioic acid. This leads to a partially oligomeric structure of the ester quats, which can have an advantageous effect on the ability of the products to dissolve to give clear solutions, particularly when co-using adipic acid. Usually, the ester quats are commercially available in the form of 50 to 90% strength by weight alcoholic solutions, which can be diluted with water as required without problems.

As component B, the conditioner according to the invention comprises a microemulsion.

Microemulsions are understood as meaning all macroscopically homogeneous, optically transparent, low viscosity and in particular thermodynamically stable mixtures of two immiscible liquids and at least one nonionic or ionic surfactant. The average particle sizes of the microemulsions are usually below 100 nm, they have a high transparency and are stable to visible phase separation upon centrifugation at 2000 rpm for at least 30 minutes.

The microemulsions are preferably produced simply by mixing the oil phase with the further oil-soluble ingredients, heating the oil phase above the melting point of all constituents and subsequently adding the aqueous surfactant-containing phase. The thermodynamically stable microemulsion is then formed spontaneously, if necessary with stirring.

The microemulsion B) present in the conditioner according to the invention comprises
b1) at least one alkyl glycoside and/or alkyl oligoglycoside,
b2) at least one cosurfactant,
b3) an organic oil phase,
b4) water.

Preferably, the microemulsion B) comprises:
b1) 4-30% by weight of an alkyl glycoside and/or alkyl oligoglycoside,
b2) 1-12% by weight of cosurfactant,
b3) 5-30% by weight of an organic oil phase,
b4) water ad 100% by weight.

All % by weight data b1) to b4) refer to the total amount of the microemulsion.

Particularly preferably, the microemulsion B) consists of:
b1) 4-30% by weight of an alkyl glycoside and/or alkyl oligoglycoside,
b2) 1-12% by weight of cosurfactant,
b3) 5-30% by weight of an organic oil phase,
b4) water ad 100% by weight.

All % by weight data b1) to b4) refer to the total amount of the microemulsion.

### APGs

The microemulsion comprises, as obligatory constituents, at least one sugar surfactant, specifically at least one alkyl glycoside and/or alkyl oligoglycoside. Within the context of the invention, the term alkyl (oligo)glycosides is used synonymously to alkyl (poly)glycosides and also referred to by the abbreviation “APG”. Alkyl glycosides and/or alkyl oligoglycosides comprise both alkyl and alkenyl (oligo)glycosides and preferably have the formula (VI)

R²³O-[G]ₚ (VI)

in which R²³ is an alkyl and/or alkenyl radical having 4 to 22 carbon atoms, G is a sugar radical having 5 or 6 carbon atoms and p is numbers from 1 to 10. They can be obtained by the relevant methods of preparative organic chemistry. The alkyl and/or alkenyl oligoglycosides can be derived from aldoses or ketoses having 5 or 6 carbon atoms, preferably glucose. The preferred alkyl and/or alkenyl oligoglycosides are thus alkyl and/or alkenyl oligoglucosides. The index number p in the general formula (VI) indicates the degree of oligomerization (DP), i.e. the distribution of mono- and oligoglycosides, and is a number between 1 and 10. Whereas p in a given compound must always be a whole number and here in particular can assume the values p=1 to 6, the value p for a specific alkyl oligoglycoside is an analytically determined calculated parameter which in most cases is a fractional number. Preference is given to using alkyl and/or alkenyl oligoglycosides with an average degree of oligomerization p of from 1.1 to 3.0. From an applications-related point of view, preference is given to those alkyl and/or alkenyl oligoglycosides whose degree of oligomerization is less than 1.7 and is in particular between 1.2 and 1.4. APGs are present in the microemulsions according to the present invention in amounts between 4 and 30% by weight, in each case based on the total amount of the microemulsion. Particular preference is given here to amounts in the range from 10 to 25% by weight.

Within the context of the invention, the cosurfactants used in the microemulsion are preferably esters of glycerol with fatty acids of chain length C₁₂-C₂₂. Preference is given here to using partial esters and in particular monoesters of glycerol, with monoesters of glycerol with unsaturated linear fatty acids being particularly suitable. Within the context of the invention, particular preference is given to glycerol monooleate. These glycerol esters are present in the microemulsions in amounts of from 1 to 12% by weight, preferably 4 to 10, in each case based on the total weight of the microemulsion.

Finally, the microemulsions also comprise an organic oil phase, i.e. a non-water-soluble organic phase, preferably in amounts of from 5 to 30% by weight. In this connection, particularly preferred organic oil phases, with the exception of alkoxylated compounds, are selected from the group which is formed by Guerbet alcohols based on fatty alcohols having 6 to 20 carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, esters of linear C₆-C₂₂-fatty acids with branched alcohols, esters of C₆-C₂₂-fatty alcohols and/or Guerbet alcohols with aromatic carboxylic acids, triglycerides based on C₆-C₁₀-fatty acids, esters of C₂-C₁₂-dicarboxylic acids with linear or branched alcohols having 1 to 22 carbon atoms or polyols having to 10 carbon atoms and 2 to 6 hydroxyl groups, vegetable oils, branched primary alcohols, substituted cyclohexanes, linear and branched C₆-C₂₂-fatty alcohol carbonates, Guerbet carbonates based on fatty alcohols having 6 to 18, preferably 8 to 10, carbon atoms, esters of benzoic acid with linear and/or branched C₆-C₂₂-alcohols n linear or branched, symmetrical or asymmetrical dialkyl ethers having 6 to 22 carbon atoms per alkyl group, and/or aliphatic and/or naphthenic hydrocarbons, dialkylcyclohexanes.

As oil phase, however, it is also possible to use solid fats and/or waxes. These may also be present in a mixture with the oils specified in the previous paragraph. Typical examples of fats are glycerides, i.e. solid or liquid vegetable or animal products which consist essentially of mixed glycerol esters of higher fatty acids. Suitable waxes are, inter alia, natural waxes, such as e.g. candelilla wax, carnauba wax, Japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microwaxes; chemically modified waxes (hard waxes), such as e.g. montan ester waxes, sasol waxes, hydrogenated jojoba waxes, and also synthetic waxes, such as e.g. polyalkylene waxes and polyethylene glycol waxes. Tocopherols and essential oils are likewise suitable as oil component.

Hydrocarbons is the term used to refer to organic compounds which consist only of carbon and hydrogen. They comprise both cyclic and acyclic (=aliphatic) compounds. They comprise both saturated and mono- or polyunsaturated compounds. The hydrocarbons may be linear or branched. Depending on the number of carbon atoms in the hydrocarbon, the hydrocarbons can be divided into uneven-numbered hydrocarbons (such as for example nonane, undecane, tridecane) or even-numbered hydrocarbons (such as for example octane, dodecane, tetradecane). Depending on the type of branching, the hydrocarbons can be divided into linear (=unbranched) or branched hydrocarbons. Saturated, aliphatic hydrocarbons are also referred to as paraffins.

Particularly preferred oil phases are ester oils such as isopropyl palmitate, isopropyl myristate, ethylhexyl palmitate, ethylhexyl stearates, di-n-octyl carbonates, dicaprylyl carbonates, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate, dioctyl malate, propylene glycol, dimerdiol or trimertriol and ether oils such as dicaprylyl ether or mixtures thereof.

A further essential constituent of the microemulsions is water. The water should preferably be demineralized. The microemulsions preferably comprise up to 80% by weight of water. Preferred ranges for the water fraction in the microemulsion are amounts of from 20 to 80% by weight and in particular from 30 to 65% by weight of water in the microemulsion. For the conditioners, a preferred water fraction of greater than 60% by weight, based on the total amount of the conditioners, arises. This means that the fraction of water from the microemulsion present is included in the 60% by weight. Similarly, water from the other ingredients, which are never free from water, is included.

Besides the ingredients described above, the conditioner also comprises, as additional constituent C), fatty alcohols of the general formula (VII)

R²⁴—OH (VII),

where R²⁴ is a saturated or unsaturated, branched or unbranched alkyl or alkenyl radical having 6 to 22 carbon atoms. Typical examples are caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof which are produced e.g. during the high-pressure hydrogenation of technical-grade methyl esters based on fats and oils or aldehydes from the Roelen oxosynthesis, and also as monomer fraction during the dimerization of unsaturated fatty alcohols. Preference is given to technical-grade fatty alcohols having 12 to 18 carbon atoms, such as, for example, coconut, palm, palm kernel or tallow fatty alcohol. Particular preference is given to the co-use of cetyl alcohol, stearyl alcohol, arachyl alcohol and behenyl alcohol, and mixtures thereof.

Fatty alcohols are preferably used in amounts of from 2 to 10% by weight, based on the conditioners, with the range from 1 to 8% by weight being particularly preferred. According to the invention, these fatty alcohols, which are water-insoluble organic constituents, do not fall under the definition of the oil phase in the microemulsion.

Besides the described cationic surfactants, the conditioner can also comprise further surfactants as component D. These further surfactants are selected from the group which is formed by anionic, nonionic, zwitterionic or amphoteric surfactants.

Typical examples of anionic surfactants are soaps, alkylbenzene sulfonates, alkane sulfonates, olefin sulfonates, α-methyl ester sulfonates, sulfo fatty acids, alkyl sulfates, monoglyceride sulfates, fatty acid amide sulfates, mono- and dialkyl sulfosuccinates, mono- and dialkyl sulfosuccinamates, sulfotriglycerides, amide soaps, ethercarboxylic acids and salts thereof, fatty acid isothionates, fatty acid sarcosinates, fatty acid taurides, N-acylamino acids, such as, for example, acyl lactylates, acyl tartrates, acyl glutamates and acyl aspartates, alkyl oligoglucoside sulfates, alkyl oligoglucoside carboxylates, protein fatty acid condensates (in particular wheat-based vegetable products) and alkyl phosphates. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants comprise polyglycol ether chains, these can have a conventional homolog distribution, but preferably have a narrowed homolog distribution.

Typical examples of amphoteric and zwitterionic surfactants are alkylbetaines, alkylamidobetaines, aminopropionates, aminoglycinates, imidazolinium-betaines and sulfobetaines. Preference is given to alkyl sulfate and particularly preferably a combination of alkyl sulfate and cocamidopropylbetaine, very particularly preferably a combination of lauryl sulfate and cocamidopropylbetaine. The anionic, nonionic, amphoteric or zwitterionic surfactants are preferably admixed in amounts of 0-5% by weight of the conditioners.

According to the invention, the cosurfactants are in particular glycerol fatty acid esters and already described above for the microemulsion. However, the glycerol monostearyl esters are preferred as further surfactant in the conditioner. If the cosurfactants are used here, they are preferably present in the conditioner with 0-5% by weight.

The sum of the further surfactants from anionic, nonionic, amphoteric or zwitterionic surfactants and cosurfactants is thus preferably between 0 and 10% by weight, calculated as active substance, based on the total amount of the conditioners.

According to the invention, the conditioner can also comprise further cosmetic additives as component E). Depending on the use of the conditioners according to the invention, further additives also have to be admixed which are selected from the group which is formed by emulsifiers, pearlescent waxes, stabilizers, salt, thickeners, consistency regulators, inorganic and organic UV photoprotective filters, self-tanning agents, pigments, antioxidants, hydrotopes, biogenic active ingredients, dyes, preservatives, preferably benzoic acid or citric acid, humectants such as glycerol, ethanol, antidandruff agents, film formers, swelling agents and perfumes. Preferred biogenic active ingredients here are in particular tocopherol, tocopherol acetate, tocopherol palmitate, deoxyribonucleic acid, coenzyme Q10, ascorbic acid, retinol and retinyl derivatives, bisabolol, allantoin, phytantriol, panthenol, AHA acids, amino acids, ceramides, essential oils, hyaluronic acid, creatine, protein hydrolyzates, plant extracts, peptides and vitamin complexes.

### EXAMPLES

1

TABLE 1
Formulation examples for conditioners

| | | | | % by | |
|---|---|---|---|---|---|
| | | | Active | weight | |
| | | | substance | in the | |
| | | | in the raw | conditioner | |
| | Substance | | material | | Comp. |
| | (raw material) | INCI | [% by wt.] | Ex. 1 | 1 |
| 1 | Plantacare ® 818 UP | Coco- | 50 | 2 | 2 |
| | | glucoside | | | |
| 2 | Dehyquart ® L80 | See below | 100 | 1 | 1 |
| 3 | Lanette ® O | Cetyl stearyl | 100 | 5 | 5 |
| | | alcohol | | | |
| 4 | Cutina ® GMS | Glyceryl | 100 | 1 | 1 |
| | | stearate | | | |
| 5 | Copherol ® 1250 C | Tocopheryl | | 0.2 | 0.2 |
| | | acetate | | | |
| 6 | Dehyquart ® A-CA | Cetrimonium | 25 | 2 | 2 |
| | | chloride | | | |
| 7 | Plantasil ® Mikro | See below | | 5.0 | — |
| 8 | Gluadin ® WLM | Hydrolyzed | 20 | 0.5 | 0.5 |
| | | wheat protein | | | |
| 9 | Water, demineralized | | | ad 100 | ad 100 |
| | Residual combability | | | 24 | 26 |
| | [%] | | | | |

Plantasil ® Mikro is a microemulsion and has the following
composition:

| | | % by wt. |
|---|---|---|
| | | of active |
| Substance | INCI | substance |
| Plantacare ® 2000 UP | Decyl glucoside | 17.5 |
| Monomuls ® 90 O 18 | Glyceryl oleate | 8 |
| Cetiol ® OE | Dicaprylyl ether | 20 |
| Demineralized water | | ad 100 |

Dehyquart ® L80 has the following INCI name: Dicocoylethyl Hydroxyethylmonium Methosulfate (and) Propylene Glycol

### 2. Preparation Procedure:

All of the components apart from the microemulsion were melted and homogenized at ca. 60° C. The microemulsion was then added and the product was stirred until it appeared homogeneous. The product was left to cool with stirring.

### 3. Determination of the Combability

The investigations as regards the conditioning performance of the conditioners were carried out in each case on 10 hair tresses in an automated system for determining wet combability.

The pretreatment of the hair tresses (dark brown European hair) (12 cm/1 g for wet combability and 15 cm/2 g hair for the dry combability) from IHIP were carried out in an automated hair treatment system:
30 min cleaning with 6% sodium lauryl ether sulfate, pH 6.5, then thorough rinsing of the hair
20 min bleaching with a solution of 5% hydrogen peroxide, pH 9.4 (adjusted with ammonium hydroxide solution), then thorough rinsing of the hair
30 min drying in a stream of air at 68° C. Directly prior to the zero measurement, the hair was swelled in water for 30 minutes and then rinsed using an automatic wet combing-out apparatus for 1 min. In the automated system for determining the wet and dry combing work, the combing forces during 20 combings were determined and the combing work was calculated by integrating the measured force-displacement curves. After the zero measurement, the hair was immediately treated with the formulation (0.125 g/g of hair). After a contact time of 3 minutes, rinsing was carried out with the automatic wet combing-out apparatus under standard conditions (38° C., 1 l/minute).

The treatment and the subsequent rinsing out was repeated a second time. The comparison measurement (for zero measurement) was then carried out. The measurements were carried out using the fine comb side of natural rubber combs. The residual combing work per tress was calculated as follows:

Residual combing work=combing work after product treatment/combing work before product treatment

The average value and the standard deviation were then determined via the quotients of all 10 tresses. The values are given in table 1.

1. A conditioner in the form of an optically non-transparent dispersion comprising:
A) at least one cationic surfactant,
B) a microemulsion comprising
b1) at least one alkyl glycoside and/or alkyl oligoglycoside,
b2) at least one cosurfactant which does not fall under the definition of b1),
b3) an organic oil phase, and
b4) water,
C) at least one fatty alcohol,
D) optionally further surfactants which do not fall under the definition of A) or C),
E) optionally further cosmetic additives which do not fall under the definition of A), C) or D),
where the sum of all of the surfactants present in the conditioner constitute a fraction of at most 10% by weight of the conditioner.

2. The conditioner of claim 1, wherein the sum of all of the surfactants present in the conditioner constitute a fraction of at most 8% by weight of the conditioner.

3. The conditioner of claim 1, wherein the conditioner comprises less than 2% by weight of alkoxylated compounds.

4. The conditioner of claim 1, wherein the conditioner comprises less than 2% by weight of silicon compounds.

5. The conditioner of claim 1, comprising:
A) 1-9.9% by weight of at least one cationic surfactant,
B) 0.1-15% by weight, preferably 0.5-10% by weight, of a microemulsion comprising:
b1) 4-30% by weight of at least one alkyl glycoside and/or alkyl oligoglycoside,
b2) 1-12% by weight, in particular 4 to 10% by weight, of at least one cosurfactant,
b3) 5-30% by weight of an organic oil phase,
b4) water ad 100% by weight based on the total amount of the microemulsion,
C) 2-10% by weight of at least one fatty alcohol,
D) 0-8.9% by weight of further surfactants,
E) 0-5% by weight of further cosmetic additives, and
F) water ad 100% by weight based on the total amount of the conditioner.

6. The conditioner of claim 1, wherein the at least one cationic surfactant is selected from the group which is formed by quaternary ammonium compounds, quaternized fatty acid trialkanolamine ester salts, specifically quaternized fatty acid triethanolamine ester salts, quaternized ester salts of fatty acids with diethanolalkylamines, quaternized ester salts of fatty acids with 1,2-dihydroxypropyldialkylamines, tetraalkylammonium salts and quaternized protein hydrolyzates.

7. The conditioner of claim 1, wherein
the at least one alkyl glycoside and/or alkyl oligoglycoside of b1) has the general formula (VI)
R²³O-[G]ₚ (VI),
in which R²³ is an alkyl and/or alkenyl radical having 4 to 22 carbon atoms, G is a sugar radical having 5 or 6 carbon atoms and p is numbers from 1 to 10.

8. The conditioner of claim 1, wherein
the cosurfactants of b2 comprise glycerol fatty acid esters.

9. The conditioner of claim 1, wherein
the organic oil phase of b3) comprises at least one compound which is selected from the group consisting of a fatty alcohol ether, a fatty acid ester of a long-chain (i.e. comprising at least 6 carbon atoms) fatty alcohol, and a dialkyl carbonate.

10. The conditioner of claim 1, wherein the at least one fatty alcohol has the general formula (VII):
R²⁴—OH (VII),
wherein R²⁴ is a saturated or unsaturated, branched or unbranched alkyl or alkenyl radical having 6 to 22 carbon atoms.

11. The conditioner of claim 1, wherein the further surfactants are selected from the group consisting of anionic, nonionic, zwitterionic, and amphoteric surfactants.

12. The conditioner of claim 1, wherein the further cosmetic additives are selected from the group consisting of emulsifiers, pearlescent waxes, stabilizers, salts, thickeners, consistency regulators, inorganic and organic UV photoprotective filters, self-tanning agents, pigments, antioxidants, hydrotopes and/or humectants such as glycerol, antidandruff agents, film formers, swelling agents, insect repellants, biogenic active ingredients, dyes, preservatives, and perfumes.

13. A method for conditioning hair, the method comprising obtaining the conditioner of claim 1, bringing the conditioner into contact with the hair, wherein the condition is effective for improving the combability of the hair.

14. The method claim 13, wherein the conditioner is brought into contact with the hair after washing the hair.

15. A method for producing the conditioner of claim 1, the method comprising:
melting components A), C), D) if present, and E) if present to form a resulting melt,
optionally homogenizing the resulting melt to form a homogenized melt, where the melting and optionally the homogenizing take place at 50 to 70° C.; and
adding component B), the microemulsion, to the resulting melt or the optionally homogenized melt.

16. The method of claim 15, wherein the microemulsion is added by stirring.
